(19) United States
(12) Patent Application Publication
(10) Pub. No.: US 2010/0105028 A1
(43) Pub. Date: Apr. 29, 2010

(54) PROBE FOR DETECTING NUCLEIC ACIDS

(75) Inventors: Jens Burmeister, Koln (DE); Oliver Seitz, Berlin (DE); Olaf Köhler, Rheinfelden (DE); Lars Röglin, Berlin (DE)

Correspondence Address: Briscoe, Kurt G.;Norris McLaughlin & Marcus, PA, 875 Third Avenue, 8th Floor, New York, NY, 10022, US

(73) Assignee: BAYER TECHNOLOGY SERVICES GMBH, Leverkusen (DE)

(21) Appl. No.: 11/722,222

(22) PCT Filed: Dec. 13, 2005
(86) PCT No.: PCT/EP05/13338

§ 371 Date: Dec. 7, 2009

(30) Foreign Application Priority Data
Dec. 23, 2004 (DE) 10 2004 063 339.8

(51) Int. Cl. C12Q 1/68; C07K 9/00; C07H 21/04; C12P 19/34

(52) U.S. Cl. 435/6; 530/322; 536/24.3; 435/91.2

(57) ABSTRACT

The invention relates to a probe for detecting nucleic acids, to a method for producing the probe, methods for carrying out analytical reactions and test kits containing the reagents that are required for carrying out the probe-based analytical reaction.

The invention relates to a probe for detecting nucleic acids, to processes for preparing said probe, to methods for carrying out detection reactions and to test kits comprising those reagents which are required for carrying out the probe-based detection reaction.

The detection of nucleic acids is widely applied, for example in human or veterinary diagnostics, in the food sector, in environmental analysis, in crop protection, in biochemical or pharmacological research, and in forensic medicine.

According to the prior art, nucleic acids are detected either by heterogeneous or homogeneous assays. Heterogeneous assays are those methods which require at least one washing step in order to separate bound and unbound probes from one another. Heterogeneous assays are carried out, for example, by having the probe immobilized to a solid phase, while the nucleic acid to be detected is in solution. Examples of detecting nucleic acids by heterogeneous assays are hybridizations to filters or DNA microarrays (see, for example, M. L. M. Anderson, Nucleic Acid Hybridization, Springer-Verlag, New York, 1998 or D. Bowtell, J. Sambrook, DNA Microarrays, Cold Spring Harbor Laboratory Press, New York, 2003). The advantage of heterogeneous assays is the intrinsic capability of simultaneously detecting a plurality of analytes (multiplexing), with a disadvantage being, for example, the requirement of washing steps. Homogeneous assays are distinguished in that all components react with one another in solution, with washing steps being completely eliminated. Homogeneous assays can usually be selected using methods with comparatively simple apparatus, but the capacity for multiplexing is usually limited. Dispensing with washing steps facilitates automation, reduces the risk of contaminations and allows the detection reactions to be carried out in a cost-effective manner.

Probes for detecting DNA usually consist of a hybridizing and a signaling unit. The hybridizing unit binds sequence-specifically to the DNA target and consists, for example, of DNA, PNA or other DNA analogs. The signaling unit may be, for example, a radiolabel, a micro- or nanoparticle, a redox-active molecule, a luminescent or a fluorescent molecule. The signaling unit is usually linked covalently to the hybridizing unit. The hybridizing unit is commonly linked to the signaling unit on the 5′ or 3′ terminus of the probe in order to prevent the signaling unit from interfering with hybridization. While heterogeneous assays use washing steps for separating probes bound to the nucleic acid to be detected and probes free in solution, homogeneous assays must ensure that the signaling unit has different properties in the hybridized state of the probe than those in the free state. A change in the signaling units following hybridization of the probe may be achieved, for example, by fluorescence resonance energy transfer (FRET) or by DNA-intercalating molecules. Examples of fluorescent, homogeneous probes which work according to the FRET principle are TaqMan® probes (P. M. Holland et al., Proc. Natl. Acad. USA, 1991, 88, 7276-7280) or “molecular beacons” (WO 95/13399 A1; S. Tyagi and F. Kramer, Nature Biotechnol. 1996, 14, 303-307). Probes working according to the FRET principle contain at their termini a fluorophore and a quencher, whereby the fluorescence can be switched, so to speak, depending on the state of hybridization. In the case of molecular beacons, the probe used is a DNA hairpin structure which is linked at its termini to a fluorophore and a quencher. In the state of no hybridization to the target, fluorescence is suppressed due to the proximity of the quencher. In the case of target binding, fluorophore and quencher are spatially separated, thereby generating a fluorescence signal whose intensity increases as a function of increasing amount of target. Molecular beacons may be used as probes in a PCR reaction in order to quantify the amount of product generated after each cycle. According to the prior art, quantitative PCR (qPCR) is one of the most common methods for detecting and quantifying nucleic acids in the research laboratory and in diagnostics. Disadvantages of molecular beacons are their intrinsic fluorescence which limits sensitivity, the limited specificity in discriminating between complementary and single-base mismatched targets and the very narrow temperature window, within which said probes can be employed. Linking specificity and sensitivity is the central challenge in the development of hybridization probes, which is of crucial importance in particular for detecting single base mutations (SNPs).

When intercalating dyes, such as, for example, SybrGreen, are added to the PCR mixture, this dye intercalates to the DNA double strand formed, resulting in an increase in fluorescence as a function of increasing amount of product. This method enables a probe-free quantitative PCR to be carried out (an example can be found in A. K. Bhar et al. J. Clin. Microbiol. 2001, 39, 2835-2845). However, a disadvantage of the method is the intrinsic fluorescence of the dye which has to be employed at a high concentration, and the completely unspecific staining. This process can also achieve only limited multiplexing of qPCR, for example by recording melting curves which can be used for discriminating different products. DNA-binding, signaling molecules whose signal properties change due to said DNA binding may likewise be utilized for developing homogeneous probes, with specificity being ensured by linking the signaling molecule to the probe. This principle was first realized by Barton et al. who linked die ruthenium(II) complexes to DNA probes (U.S. Pat. No. 5,157,032; Y. Jenkins et al., Biochemistry 1992, 31, 10809-10816). These probes exhibit an increase in fluorescence upon hybridization. However, fluorescence also increases upon addition of double-stranded non-target DNA. The cationic ruthenium complex moreover favors unspecific hybridizations and substantially restricts the specificity of the probes. EP 0 710 668 B1 describes detection of nucleic acids by using DNA probes linked at their termini to an asymmetric cyanine dye. Hybridization of these probes produces a small increase in fluorescence, by about a factor of 4, which does not enable sensitive detection of nucleic acids. U.S. Pat. No. 6,329,144 B1 describes a PNA probe which is linked at its termini to an asymmetric cyanine dye. This LightUp® probe exhibits a distinct increase in fluorescence upon hybridization to nucleic acids. These probes meet the criterion of sensitivity, which was also demonstrated in the context of qPCR (N. Svanvik et al., Anal. Biochemistry 2000, 287, 179-182). However, regarding specificity, the LightUp® probes are limited to the discriminating capacity of the PNA sequence itself, as a result of which only a limited difference in fluorescence intensities between matching and non-matching base pairs can be achieved in the detection of single base mutations (an example is the difficult discrimination of G:C and G:T base pairs).

Privat et al. have described DNA probes in which a central, internucleotidic phosphate group was linked via a linker to thiazole orange (E. Privat et al., Photochem. Photobiol. 2002, 75, 201-210). These probes exhibited only a low increase in fluorescence, by a factor of five, when hybridizing complementary targets. Owing to the resulting, low detection sensitivity, the probes described are not suitable for practical application in nucleic acid diagnostics.

A DNA probe for homogeneous detection of nucleic acids on the basis of fluorescent nucleosides has been described by Okamoto et al. (A. Okamoto et al., J. Am. Chem. Soc. 2003, 125, 9296-9297). The fluorescent nucleosides incorporated into the probes were methoxybenzodeazaadenine (^{MD}A) and methoxybenzodeazainosine (^{MD}I), with ^{MD}A producing distinct fluorescence in the presence of C in the counter strand, while ^{MD}I produces distinct fluorescence in the presence of T in the counter strand. These probes are suitable in principle for discriminating single base mutations in homogeneous assays, but a disadvantage is the fact that fluorescence of the fluorescent nucleosides is suppressed by flanking G:C base pairs (fluorescence quenching). This property markedly restricts the general applicability of these probes. WO2004058793 A1 discloses a similar principle: the nucleobases uracil and cytosine were linked via a propargyl linker to pyrene carboxamide and the fluorescent bases obtained in this way (^{Py}U and ^{Py}C) were integrated into DNA probes. Although the matching base pairs ^{Py}U:A and ^{Py}C:G exhibit a distinctly higher fluorescence than the more weakly pairing combinations, hybridization of the ^{Py}U and ^{Py}C probes results in only small increases in fluorescence (2-8 fold). Another disadvantage of this process is the fact that only one fluorescence dye is available and therefore assay multiplexing cannot be implemented.

Köhler and Seitz demonstrated that the fluorescence dye thiazole orange can be attached as a universal base surrogate to a PNA probe with aminoethylglycine backbone and thus enables a PNA probe having improved specificity to be designed (O. Köhler, O. Seitz, Chem. Commun 2003, 23, 2938-2939). When single base mutations are detected by these probes, the universal base thiazole orange is introduced next to the site of mutation. The environmentally sensitive dye produces a maximum fluorescence signal only if it is in the vicinity of perfect base pairs. Experiments with this probe revealed that the perfectly paired duplex of the probe and a target complementary thereto exhibited more fluorescence than the duplex containing a mismatch even when hybridization took place below the duplex melting point. This observation indicates that thiazole orange enables the probes to achieve a specificity which exceeds the specificity of the complementary base pairing. While the specificity of these probes on the basis of a PNA sequence with aminoethylglycine backbone is an improvement over the prior art, the fluorescence amplification factor achieved is comparable to other homogeneous probes.

Improving specificity and sensitivity is the central problem in the development of hybridization probes, which is of crucial importance in particular for detection of single base mutations (SNPs). In addition, the probes should be usable over as wide a temperature range as possible and enable assay multiplexing.

It is therefore the object of the present invention to provide a probe for detecting nucleic acids which hybridizes with high specificity and selectivity and over a very large temperature range, can be used in homogeneous assays and allows multiplex detections.

Surprisingly, it turned out that there is a probe which solves said object.

The invention therefore relates to a probe for detecting nucleic acids which consists of a peptide nucleic acid strand, a nucleic acid strand or a strand of DNA analogs, in which strand a nucleobase at an internal position has been replaced by a fluorescent base surrogate. Surprisingly, it was found that, for example, thiazole orange which had been bound to ornithine exhibited a distinctly higher fluorescence amplification upon hybridization to a target sequence without mismatch than thiazole orange which had been attached according to the prior art to an aminoethylglycine backbone. Another surprising observation was the fact that L-ornithine and D-ornithine conjugates differ with respect to the ratio of specificity of hybridization to achievable fluorescence amplification: the L-ornithine conjugate exhibited a higher fluorescence amplification than the D-ornithine conjugate, while the D-ornithine conjugate exhibited a higher specificity of said fluorescence amplification.

The invention therefore relates to a PNA probe, available by solid phase synthesis, of the general formula:

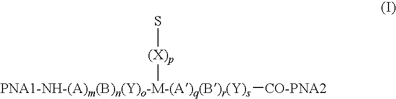

in which

PNA1 is a peptide nucleic acid strand of any sequence and length, preferably a sequence of 2-100 bases, particularly preferably between 2-10 bases.

PNA2 is a peptide nucleic acid strand of any sequence and length, preferably a sequence of 2-100 bases, particularly preferably between 2-10 bases.

A and A′ are independently of one another a methylene group or a substituted carbon moiety of the CHR or CR2 type, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, a substituted or unsubstituted phenyl group, particularly preferably the in each case unsubstituted and unbranched embodiments and very particularly preferably hydrogen or a methyl group, with A preferably being a methylene group.

B and B′ are independently of one another a methylene group or a substituted carbon moiety of the CHR or CR2 type, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, a substituted or unsubstituted phenyl group, particularly preferably the in each case unsubstituted and unbranched embodiments and very particularly preferably hydrogen or a methyl group, with B preferably being a methylene group.

m is a natural number from 1-5, preferably 1,

n is a natural number from 1-5, preferably 1,

o is a natural number from 1-5, preferably 1,

X is a methylene group or a substituted carbon moiety of the CHR or CR2 type, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, or a substituted or unsubstituted phenyl group, or

X is a substituted or unsubstituted amino group of the NH or NR type, wherein R is hydrogen or an organic rest, preferably a substituted or unsubstituted branched or unbranched C1-C10 alkyl group—wherein the substituents may be chosen preferably from the group consisting of elements of the 5th or 6th main group, preferably O or S—, a substituted or unsubstituted phenyl group,

or X is an atom of the 5th or 6th main group of the Periodic Table, preferably oxygen or sulfur,

X is particularly preferably an NH group.

p is a natural number from 0 to 10, preferably a number from 1-6,

S is a fluorescent, universal base surrogate, characterized in that it is fluorescent and able to intercalate into DNA, preferably a cyanine dye, furthermore preferably ethidium, furthermore preferably a substituted anthracene, furthermore preferably an acridine dye, furthermore preferably a dye selected from the dyes sold by Molecular Probes, PO-PRO-1, BO-PRO-1, YO-PRO-1, TO-PRO-1, JO-PRO-1, PO-PRO-1, PO-PRO-3, LO-PRO-1, BO-PRO-3, YO-PRO-3, TO-PRO-3, TO-PRO-5 (and fluorophores derived from these compounds, which have a modified linker structure), particularly preferably thiazole orange,

M is a substituted carbon moiety of the CH or CR type (wherein R is hydrogen, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, or a substituted or unsubstituted phenyl group, or

M is a substituted or unsubstituted amino group of the NH or NR type, wherein R is hydrogen or an organic rest, preferably a substituted or unsubstituted branched or unbranched C1-C10 alkyl group—wherein the substituents may be chosen preferably from the group consisting of elements of the 5th or 6th main group, preferably O or S—, a substituted or unsubstituted phenyl group,

or M is an atom of the 5th or 6th main group of the Periodic Table, preferably oxygen or sulfur,

M is particularly preferably a CH group.

Y is defined as A.

q is a natural number from 0-5, preferably 0,

r is a natural number from 0-5, preferably 0,

s is a natural number from 0-5, preferably 0.

The hybridizing oligomer of the nucleic acid type may, as an alternative to PNA, also be DNA or a DNA analog such as, for example, LNA, morpholino-DNA or arabino-DNA. Accordingly, the invention also relates to probes, obtainable by solid phase synthesis, of the general formula (II):

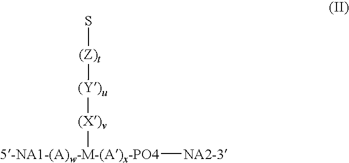

in which

5′-NA1 is a nucleic acid or a nucleic acid analog of any sequence and length, which ends in a 3′-terminal phosphate group, preferably a nucleic acid sequence of 3-100 bases in length, particularly preferably a nucleic acid sequence of 3-20 bases in length, very particularly preferably a nucleic acid sequence of 3-15 bases in length,

NA2-3′ is a nucleic acid or a nucleic acid analog of any sequence and length, preferably a nucleic acid sequence of 3-100 bases in length, particularly preferably a nucleic acid sequence of 3-20 bases in length, very particularly preferably a nucleic acid sequence of 3-15 bases in length,

A and A′ are as defined above.

w and x are a natural number from 0-5,

X′ and Z are independently of one another a methylene group or a substituted carbon moiety of the CHR or CR2 type, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, or a substituted or unsubstituted phenyl group, or

X′ and Z are independently of one another a substituted or unsubstituted amino group of the NH or NR type, wherein R is hydrogen or an organic rest, preferably a substituted or unsubstituted branched or unbranched C1-C10 alkyl group—wherein the substituents may be chosen preferably from the group consisting of elements of the 5th or 6th main group, preferably O or S—, a substituted or unsubstituted phenyl group, or

X′ and Z are independently of one another an atom of the 5th or 6th main group of the Periodic Table, preferably oxygen or sulfur,

X′ and Z are independently of one another particularly preferably a methylene group.

Y′ is a methylene group or a substituted carbon moiety of the CHR or CR2 type, wherein R is hydrogen or an organic rest, preferably a substituted or unsubstituted branched or unbranched C1-C10 alkyl group—wherein the substituents may be chosen preferably from the group consisting of substituted or unsubstituted amino groups, elements of the 5th or 6th main group, preferably O or S—, or a substituted or unsubstituted phenyl group, or

Y′ is a substituted or unsubstituted amino group of the NH or NR type, wherein R is hydrogen or an organic rest, preferably a substituted or unsubstituted branched or unbranched C1-C10 alkyl group—wherein the substituents may be chosen preferably from the group consisting of elements of the 5th or 6th main group, preferably O or S—, a substituted or unsubstituted phenyl group,

or Y′ is an atom of the 5th or 6th main group of the Periodic Table, preferably oxygen or sulfur,

Y′ is particularly preferably an NH group.

v is a natural number from 0 to 10, preferably a number from 1-6, particularly preferably 1,

u is a natural number from 0 to 10, preferably a number from 1-6, particularly preferably 1,

t is a natural number from 0 to 10, preferably a number from 1-6,

S and M are as defined above.

Preference, particular preference or very particular preference is given to embodiments making use of the parameters, compounds, definitions and illustrations listed as preferred, particularly preferred or very particularly preferred.

The definitions, parameters, compounds and illustrations listed in the description, in general or within areas of preference, however, may also be combined with one another as desired, i.e. between the particular areas and areas of preference.

The fluorescent, universal base surrogate S of the invention, as specified in the probes according to formula (I) and (H), is characterized in that it is capable of intercalating in DNA and, independently of the base opposite the surrogate in the nucleic acid target, produces the same or a similar melting temperature for the DNA-probe duplex. In this respect, the base surrogate may be referred to as universal. Examples of base surrogates of the invention are cyanine dyes such as, for example, thiazole orange, oxazole yellow (EP 0 714 986 Al), ethidium, Dapoxyl®, 2,6-donor-acceptor-substituted anthracenes (H. Ihmels et al., Org. Lett. 2000, 2, 2865-2867), acridines (see, for example, K. Fukui et al., Bioconjugate Chem. 1996, 7, 349-355), dyes sold by Molecular Probes (see Molecular Probes manual: Handbook of Fluorescent Probes and Research Products), such as, for example, PO-PRO-1, BO-PRO-1, YO-PRO-1, TO-PRO-1, JO-PRO-1, PO-PRO-1, PO-PRO-3, LO-PRO-1, BO-PRO-3, YO-PRO-3, TO-PRO-3, TO-PRO-5 (and compounds derived from these fluorophores with a modified or missing linker structure) and the BTCS chromophore (the BTCS chromophore is described, for example, in R. B. Thompson et al., J. Biomed. Opt. 2000, 5, 1, 17-22). Probes which differ in their sequence may be color-labeled, so to speak, by using base surrogates which differ with regard to their fluorescence excitation and fluorescence emission wavelength, thereby enabling a plurality of analytes to be detected simultaneously (multiplexing).

The fluorescent base surrogate is attached to a backbone which produces a longer distance between the nucleobases flanking said base surrogate than a backbone imitating the distance of neighboring nucleobases in the DNA. Examples of a backbone imitating the DNA standard geometry is the aminoethylglycine backbone in PNA. The more the distance between the fluorescent base surrogate and the flanking nucleobases deviates from the standard geometry of the DNA backbone, the less efficiently can base-stacking interactions increase the emission of the fluorescent base surrogate in the single strand. As a result, fluorescence of the probe single strand is low. However, the chosen distance must not be so large that base stacking would be prevented in the double strand produced after hybridization. A suitable distance between the fluorescent base surrogate and the flanking nucleobases within a PNA probe with aminoethylglycine backbone may be produced, for example, by introducing 3-10 methylene groups at the backbone position carrying the base surrogate. The backbone structure carrying the base surrogate may include, in addition to carbon moieties, also heteroatoms such as, for example, oxygen, nitrogen, sulfur, phosphorus or silicon. An example of a backbone of the invention at the site of the fluorescent base surrogate is ornithine within a PNA probe with aminoethylglycine backbone.

If a plurality of units of the general structure (I) or (II) are covalently linked to one another, a probe with multiple labels may be obtained. Such a procedure increases the detection sensitivity when using the probes of the invention. In contrast to terminal fluorescent labels which make possible a maximum of two labels per probe, incorporation of the fluorescent base surrogates makes multiple labeling possible.

The probe of the invention is suitable for carrying out hybridization assays, in particular homogeneous hybridization assays. The probe may be used in homogeneous hybridization assays, for example, in order to determine the type and the amount of a target nucleic acid present in an aqueous sample. To this end, the probe is added to the dissolved target nucleic acid and incubated for a few seconds up to several hours. Subsequently, the fluorescence of the solution is measured in comparison with a control (same solution without target nucleic acid). Comparing the fluorescence value with a standard curve in which the correlation between the fluorescence of the probe and the amount of target used has been determined enables quantification of the nucleic acid to be detected. If the quantitative detection of nucleic acids is intended to be carried out with a particularly high sensitivity, it is possible to combine the singly or multiply labeled probe in a particularly advantageous manner with the PCR process. During quantitative PCR, the increase in fluorescence due to target amplification is determined. The number of PCR cycles required for exceeding a predetermined threshold of fluorescence is defined as “cycle threshold” (CT). Via a calibration curve, the CT value can be related to the target concentration used and thus represents a measure of said target concentration. The probe must be chosen here so as to be complementary to a target sequence section located between the primer sequences and to exhibit as little cross hybridization as possible with other sequences of the genome.

The probe is suitable for detecting single base mutations (SNPs), which detection may be carried out, for example, by way of hybridization or PCR. It is also possible to use the probe for analyzing methylation patterns. If the DNA to be studied is treated with sodium bisulfite, all non-methylated cytosines are converted to uracil, while methylated cytosines emerge unchanged from the reaction. The resulting, partially modified target may then be analyzed by assays such as, for example, PCR, quantitative PCR or hybridization. Such a process using PCR and quantitative PCR is described, for example, in J. G. Herman et al., Proc. Natl. Acad. Sci. USA 1996, 93, 9821-9826 et al., and in T.-S. Wong et al., European Journal of Cancer 2003, 39, 1881-1887, respectively. In order to analyze DNA treated with sodium bisulfite, the probes may be used in assays, preferably in PCR or in quantitative PCR.

The probe is prepared by chemical synthesis, preferably by solid phase synthesis, particularly preferably by automated solid phase synthesis. Methods of preparing nucleic acids, nucleic acid analogs and peptide nucleic acids are known to the skilled worker and are described, for example, in L. M. Smith, Anal. Chem. 1988, 60, 381-390 and in B. E. Hyrup and P. E: Nielsen, Bioorg. Med. Chem. 1996, 4, 5-23. The probes may be linked covalently to the fluorescent base surrogate following solid phase synthesis, or the base surrogate is already incorporated during convergent synthesis.

Ornithine may be protected, for example, N-terminally by a fluorenylmethoxycarbonyl group (Fmoc group), while the base surrogate is covalently linked to the primary amino group of ornithine. For example, thiazole orange may be bound via a carboxyl group to the primary amino group of ornithine. Such a building block is suitable for use in automated solid phase syntheses of the probes by the Fmoc process.

The probe of the invention has the following advantages over the probes disclosed in the prior art for detecting nucleic acids:
a) High specificity of recognition between the probe and the nucleic acid to be detected, which is particularly advantageous when detecting single base mutations (SNPs) or methylated cytosines.
b) Low fluorescence in the single strand due to expansion of the backbone and, as a result thereof, elevated fluorescence increases upon formation of the probe-analyte complexes.
c) Possibility of generating probes having a particularly high fluorescence amplification (=detection sensitivity) or specificity by selecting the backbone.
d) The probe may be multiply labeled by incorporating a plurality of base surrogates.
e) Use in multiplex assays by way of utilizing base surrogates whose fluorescence peaks are at different wavelengths.
f) Hybridization and single base-specific discrimination within a wide temperature range under nonstringent hybridization conditions. This facilitates considerably probe design and assay optimization.
g) The probes are prepared by conventional solid phase synthesis.

The invention will be illustrated in more detail below on the basis of embodiments and figures in which:

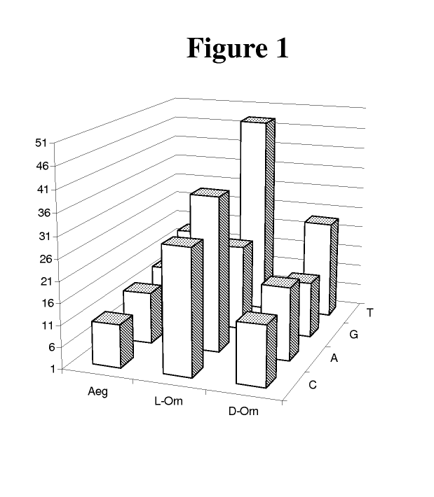

FIG. 1 depicts the ratio of the fluorescence intensities of the PNA probe Ac-GCCGTA-R(TO)-TAGCCG (sequence no. 1) after and before addition of the DNA target 5′-CGGCTAZTACGGC-3′ (sequence no. 2) as a function of the backbone R to which thiazole orange (TO) has been bound and as a function of the nucleotide opposite the base surrogate, where Z=1=thymidine (T), 2=guanosine (G), 3=adenosine (A) and 4=cytidine (C), in the duplex, wherein 5=R=aminoethylglycine (Aeg), 6=R=L-ornithine (L-Orn) and 7=R=D-ornithine (D-Orn).

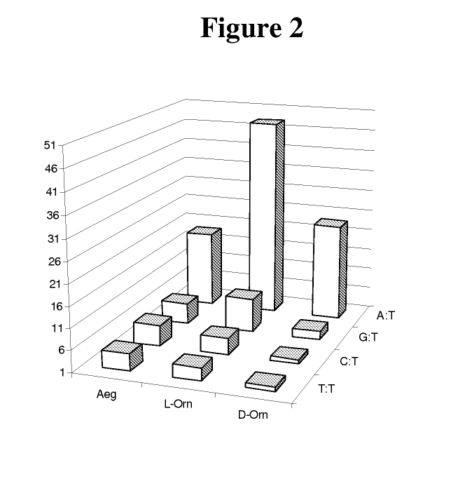

FIG. 2 depicts the ratio of the fluorescence intensities of the PNA probe Ac-GCCGTA-R(TO)-TAGCCG (sequence no. 1) after and before addition of the DNA target 5′-CGGCTYTTACGGC-3′ (sequence no. 3) as a function of the backbone R to which thiazole orange (TO) has been bound and as a function of the single base mutation present in the target, where Y=8=adenine (A), 9=guanosine (G), 10=cytidine (C) and 11=thymidine (T), wherein 5=R=aminoethylglycine (Aeg), 6=R=L-ornithine (L-Orn) and 7=R=D-ornithine (D-Orn).

### EXAMPLES OF SYNTHESIZING PROBES

The PNA monomers were purchased from PerSeptive Biosystems and dried under medium vacuum prior to synthesis. The solid phase NovaSyn® TGR, PyBOP and D-ornithine hydrochloride were purchased from Novabiochem, and L-ornithine hydrochloride was purchased from Avocado Research Chemicals. The DNA oligonucleotides were obtained from MWG-Biotech. All other chemicals were obtained from Acros, Aldrich, Avocado, Fluka and Riedel de Haen. The solvents used were, where appropriate, distilled or dried by standard processes prior to use. All aqueous solutions were prepared with water which had been purified by means of a Milli-Q-Pore apparatus (Millipore).

The probes may be synthesized using a divergent or a convergent (linear) strategy. Synthesis of the probes by applying a linear synthesis strategy will be described below, since the latter can be fully automated. In an exemplary embodiment of the invention, the Fmoc-protected thiazole orange ornithines 1 and 2 were synthesized.

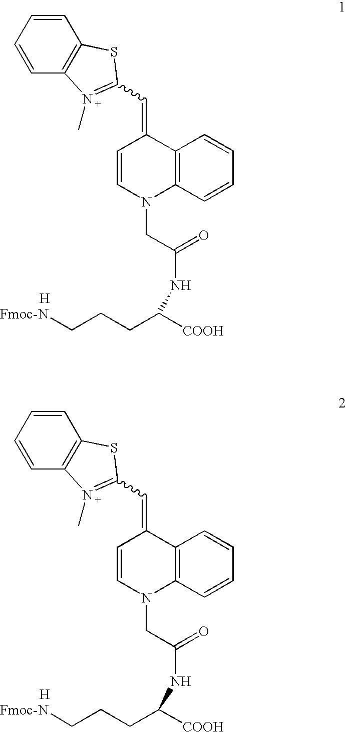

### Example 1

### Preparation of Nδ-[9-(fluorenylmethoxycarbonyl)-L-ornithine]-Nα[2-(1-carboxymethyl-1H-quinolin-4-ylidenemethyl)-3-methylbenzothiazol-3-ium bromide]allyl ester (Fmoc-L-Orn(TO)-OAll)

Thiazole orange carboxylic acid was prepared according to a protocol by Zhou et al. (X.-F- Zhou te 1., Journal of Imaging Science and Technology, 1995, 39, 244-252). A solution of thiazole orange carboxylic acid (100 mg, 0.23 mmol) in dry DMF (2.3 ml) was admixed with PyBOP (145 mg, 0.279 mmol), pyridinium p-toluene sulfonate (58 mg, 0.232 mmol) and N-methylmorpholine (23 mg, 0.23 mmol) The suspension was stirred under an argon atmosphere, until the clear solution A was obtained. L-Ornithine was protected N-terminally with Fmoc and C-terminally with an allyl group according to standard processes (=Fmoc-L-Orn-OAll). The clear solution A was added to a solution of Fmoc-L-Orn-OAll (100 mg, 0.23 mmol) and N-methylmorpholine (23 mg, 0.23 mmol) in DMF (2.3 ml). The reaction mixture was stirred under an argon atmosphere for 12 h. The solvent was removed under vacuum, the residue was taken up in methanol (2 ml). After stirring for 1 h, the solid was filtered off and then washed with methanol (5 ml). The crude product was purified by column chromatography (chloroform:methanol 97:3, 1% formic acid). 118 mg of Fmoc-L-Orn(TO)-OAll were obtained in the form of an orange solid, corresponding to a yield of 63%.

Rf (chloroform:methanol 80:20, 1% formic acid)=0.56

### Example 2

### Preparation of Nδ-[9-(fluorenylmethoxycarbonyl)-L-ornithine]-Nα-[2-(1-carboxymethyl-1H-quinolin-4-ylidenemethyl)-3-methylbenzothiazol-3-ium bromide] OH (Fmoc-L-Orn(TO)-OH) 1

A solution of Fmoc-L-Orn(TO)-OAll (81 mg, 01 mmol) in THF (5 ml) was admixed with N-methylaniline (11.3 mg, 0.1 mmol), and the solution was degassed several times. After addition of [Pd(PPh₃)₄] (12 mg, 0.01 mmol), the mixture was stirred for 14 h with exclusion of light. The solvent was removed in vacuo. The residue was dissolved in methanol (4 ml) and the precipitate was filtered off. The crude product was purified by column chromatography (chloroform:methanol 97:3, 1% formic acid). 53 mg of the product Fmoc-L-Orn(TO)-OH (1) were obtained in the form of an orange solid, corresponding to a yield of 68%.

Rf (chloroform:methanol 80:20, 1% formic acid)=0.22

### Example 3

### Preparation of Nδ-[9-(fluorenylmethoxycarbonyl)-D-ornithine]-Nα-[2-(1-carboxymethyl-1H-quinolin-4-ylidenemethyl)-3-methylbenzothiazol-3-ium bromide]allyl ester (Fmoc-D-Orn(TO)-OAll)

Thiazole orange carboxylic acid was prepared according to a protocol by Zhou et al. (X.-F- Zhou te 1., Journal of Imaging Science and Technology, 1995, 39, 244-252). A solution of thiazole orange carboxylic acid (100 mg, 0.23 mmol) in dry DMF (2.3 ml) was admixed with PyBOP (145 mg, 0.279 mmol), pyridinium p-toluene sulfonate (58 mg, 0.232 mmol) and N-methylmorpholine (23 mg, 0.23 mmol) The suspension was stirred under an argon atmosphere, until the clear solution A was obtained. L-Ornithine was protected N-terminally with Fmoc and C-terminally with an allyl group according to standard processes (=Fmoc-D-Orn-OAll). The clear solution A was added to a solution of Fmoc-D-Orn-OAll (100 mg, 0.23 mmol) and N-methylmorpholine (23 mg, 0.23 mmol) in DMF (2.3 ml). The reaction mixture was stirred under an argon atmosphere for 12 h. The solvent was removed under vacuum, the residue was taken up in methanol (2 ml). After stirring for 1 h, the solid was filtered off and then washed with methanol (5 ml). The crude product was purified by column chromatography (chloroform:methanol 97:3, 1% formic acid). 103 mg of Fmoc-D-Orn(TO)-OAll were obtained in the form of an orange solid, corresponding to a yield of 55%.

Rf (chloroform:methanol 80:20, 1% formic acid)=0.56

### Example 4

### Preparation of Nδ[-9-(fluorenylmethoxycarbonyl)-D-ornithine]-Nα-[2-(1-carboxymethyl-1H-quinolin-4-ylidenemethyl)-3-methylbenzothiazol-3-ium bromide] OH (Fmoc-D-Orn(TO)-OH) 2

A solution of Fmoc-D-Orn(TO)-OAll (81 mg, 01 mmol) in THF (5 ml) was admixed with N-methylaniline (11.3 mg, 0.1 mmol), and the solution was degassed several times. After addition of [Pd(PPh₃)₄] (12 mg, 0.01 mmol), the mixture was stirred for 14 h with exclusion of light. The solvent was removed in vacuo. The residue was dissolved in methanol (4 ml) and the precipitate was filtered off. The crude product was purified by column chromatography (chloroform:methanol 97:3, 1% formic acid). 52 mg of the product Fmoc-D-Orn(TO)-OH (1) were obtained in the form of an orange solid, corresponding to a yield of 65%.

Rf (chloroform:methanol 80:20, 1% formic acid)=0.22

### Example 5

### Solid Phase Synthesis of Probes

Loading the NovaSyn® TGR Solid Phase:

The solid phase (500 mg, 0.29 mmol/g) was washed (3× dichloromethane, 3× dimethylformamide, 3× dichloromethane, 3× dimethylformamide). The solid phase was left swelling in dimethylformamide (10 ml) for 30 min For preactivation, (benzotriazol-1-yloxy)-tripyrrolidinophosphoniumhexafluorophosphate (PyBOP® 301.2 mg, 0.58 mmol) and N-methyl-morpholine (87.7 mg, 0.87 mmol) were added to a solution of Fmoc-protected glycine (172.3 mg, 0.58 mmol) in dimethylformamide (5.8 ml). After incubating for 3 min, the solution obtained was mixed with the solid phase. After incubating for another 4 h, the solid phase was washed (3× dimethylformamide, 3× dichloromethane, 3× dimethylformamide), and groups which had not reacted were blocked with a solution of acetic anhydride in pyridine (1:4, 5 ml). The blocking step was repeated after 5 min The solid phase was washed (3× dimethylformamide, 3× dichloromethane, 3× dimethylformamide and 5× dichloromethane) and finally dried in vacuo.

Synthesis Using an Automated Synthesizer

The probes were synthesized on an automated synthesizer (Intavis ResPep from Intavis AG) according to the following general protocol:

The loaded solid phase was left swelling in dimethylformamide (2 ml). After 30 min, the swollen solid phase was transferred to the synthesizer and washed 2× with dimethylformamide (180 μl).

Fmoc removal: The solid phase was mixed with dimethylformamide/piperidine (4:1, 100 μl). The procedure was repeated after 2 min, and finally the solid phase was washed with DMF (1×180 μl, 3×100 μl and 1×180 μl).

Coupling: The solid phase was suspended in a solution of 4 equivalents of the Fmoc-protected building block to be coupled and 8 equivalents of N-methylmorpholine in dimethylformamide (concentration of the building block: 0.3M in NMP, preactivated for 8 min by incubation with 3.6 equivalents of 1H-benzotriazolium-1-[bis(dimethylamino)methylene] 5-chlorohexafluorophosphate (HCTU)). The solid phase was washed after 30 min (2×180 μl of dimethylformamide).

Capping: Capping was carried out by way of treatment with acetic anhydride/2,6-lutidine/pyridine (5:6:89, 100 μ) for 3 min. Subsequently, the solid phase was washed (2×180 μl of dimethyl-formamide, 1×100 μl of dimethylformamide).

Coupling of compounds 1 and 2 was carried out according to the protocol specified above, with PPTS (2 mg, 8 μmol) being added in order to improve solubility. Said coupling was carried out twice.

Removal from the solid phase: A solution of cystine methyl ester hydrochloride (5 mg, 29 μmol) in trifluoroacetic acid/m-cresol/water (37:2:1, 1 ml) was passed through the previously dried solid phase for 40 min. The solid phase was washed with trifluoroacetic acid (500 μl). The combined filtrates were concentrated in vacuo.

Purification: Cold diethyl ether was added to the concentrated removal solution. The precipitate was centrifuged and the supernatant was discarded. The residue was taken up in water and prepurified using a SepPak® C18 column which was equilibrated with water. The colored eluates obtained after gradient elution (1×20:80 acetonitrile:H₂O:0.1% trifluoroacetic acid; 1×40:60 acetonitrile:H₂O:1% trifluoroacetic acid; 1×80:20 acetonitrile:H₂O:0.1% trifluoroacetic acid; 1×80:20 acetonitrile:H₂O:0.1% trifluoroacetic acid; 2 ml each) were analyzed by means of HPLC and MALDI-TOF mass spectrometry and purified by semi-preparative HPLC.

### EXAMPLES OF USING THE PROBES

The following two examples demonstrate, by way of example, the way in which the probes can be used for homogeneous DNA detection and show a comparison with the prior art.

### Example 6

### Homogeneous DNA Detection with Thiazole Orange-L-Ornithine and Thiazole Orange-D-Ornithine Probes in Comparison with Thiazole Orange-Aminoethylglycine Probes

The fluorescence spectra were recorded at 25° C. using a spectrometer LS 50B from Perkin-Elmer. The measurements were carried out in phosphate buffer, pH 7 (100 mM NaCl, 10 mM Na₂HPO₄, 0.1 mM EDTA). In each case 1 nmol of the DNA target and of the particular PNA probe in 1 ml of buffer solution were used for hybridization. The PNA probe used had the general sequence Ac-gccgta-O-tagccg-Gly-NH2 (sequence no. 4), where O is the thiazole orange dye which had been coupled to an aminoethylglycine, D-ornitine or L-ornithine backbone. The DNA target had the general sequence 5′-CGGCTAXTACGGC (sequence no. 5), wherein X was the nucleobase opposite the base surrogate in the duplex, i.e. C, T, A or G. FIG. 1 depicts the ratio of the measured fluorescence intensities of the probes before and after addition of the DNA targets as a function of the backbone structure to which thiazole orange was bound and as a function of the base opposite the base surrogate. The results demonstrate by way of example that in all cases the L-ornithine backbone produces a higher fluorescence signal than the aminoethylglycine backbone. In all four cases, the D-ornithine backbone produces weaker fluorescence signals than the L-ornithine backbone. In comparison with the aminoethylglycine backbone, the D-ornithine backbone produces a higher fluorescence intensity than the aminoethylglycine backbone in three cases, with the intensities being equal in one case. This example also demonstrates that an appropriate selection of the probe sequence can maximize the fluorescence increase upon hybridization.

### Example 7

### Detection of Single Base Mutations with Thiazole Orange-L-Ornithine and Thiazole Orange-D-Ornithine Probes in Comparison with Thiazole Orange-Aminoethylglycine Probes

The fluorescence spectra were recorded at 25° C. using a spectrometer LS 50B from Perkin-Elmer. The measurements were carried out in phosphate buffer, pH 7 (100 mM NaCl, 10 mM Na₂HPO₄, 0.1 mM EDTA). In each case 1 nmol of the DNA target and of the particular PNA probe in 1 ml buffer solution were used for hybridization. The PNA probe used had the general sequence Ac-gccgta-O-tagccg-Gly-NH2 (sequence no. 4), where O is the thiazole orange dye which had been coupled to an aminoethylglycine, D-ornithine or L-ornithine backbone. The DNA target had the general sequence 5′-CGGCTXTTACGGC (sequence no. 6), wherein X was the single base mutation in the DNA target, i.e. C, T, A or G. X=A is the perfectly paired duplex. FIG. 2 depicts the ratio of the measured fluorescence intensities of the probes after and before addition of the DNA targets as a function of the backbone structure to which thiazole orange had been bound and as a function of the single base mutation present in the target. The results demonstrate that ornithine probes generally have higher specificity in the recognition of single base mutations than aminoethylglycine probes. The D-ornithine probes generally exhibit weaker fluoresce signals than the L-ornithine probes. While the signal ratio of the perfectly paired duplex and the various duplices containing a mismatch is 6-12 in the case of L-ornitine, a ratio of 8-12 is observed for D-ornithine. The example demonstrates the generally higher specificity of D-ornithine probes.

### Example 8

### Determination of Melting Temperatures of Various Duplexes with Thiazole Orange-Aminoethylglycine, Thiazole Orange-L-Ornithine and Thiazole Orange-D-Ornithine Probes

The melting curves of the probe-DNA-complexes were recorded in water using a UV-VIS spectrometer from Varian (Cary 100). The measurement was carried out using a gradient of 0.3° C. per minute, with two measurements being carried out in each case from 85° C.-15° C. and 15° C.-85° C. The point of inflexion of the melting curve was determined as the melting temperature of the particular duplex. Two DNA targets having the sequence 5′-AGTGAAATGTTATACGAAACT-3′ (sequence no. 7) (match target) and 5′-GAAATGCTATACGAA-3′ (sequence no. 8) (mismatch target) were used. The probes used were compounds of the general sequence N-ttcgtat-O-acatttc-Lys-C (sequence no. 9), wherein O is the thiazole orange dye bound to an aminoethylglycine, D-ornitine or L-ornithine backbone. The following melting temperatures were determined for the various duplexes:

| | Aminoethylglycine | L-Ornithine | D-Ornithine |
|---|---|---|---|
| Tm (match) | 59° C. | 59° C. | 55° C. |
| Tm (mismatch) | 37° C. | 38° C. | 35° C. |
| ΔTm | 22° C. | 20° C. | 21° C. |

The example demonstrates that the duplexes were not significantly destabilized by the modified (expanded) backbone structures in the case of the ornithine probes. Moreover, the differences in the melting temperatures of match and mismatch duplexes are identical within the experimental margins of error. This example demonstrates that the improved distinction between match and mismatch duplexes due to the ornithine backbone can be attributed to the change in environment of the base surrogate rather than to altered melting points. This makes it possible for the probes to be applied for single base distinction within a wide temperature range.

1. A PNA probe of the formula: 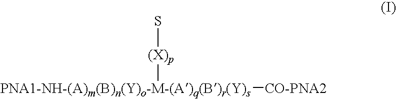
in which
PNA1 is a peptide nucleic acid strand of any sequence and length,
PNA2 is a peptide nucleic acid strand of any sequence and length,
A and A′ are independently of one another a methylene group or a substituted carbon moiety of the CHR or CR2 type, wherein R is hydrogen or an organic group,
B and B′ are independently of one another a methylene group or a substituted carbon moiety of the CHR or CR2 type, wherein R is hydrogen or an organic group,
m is a natural number from 1-5,
n is a natural number from 1-5,
o is a natural number from 1-5,
X is a methylene group or a substituted carbon moiety of the CHR or CR2 type, wherein R is hydrogen or an organic group, or
X is a substituted or unsubstituted amino group of the NH or NR type, wherein R is hydrogen or an organic group, or
X is an atom of the 5th or 6th main group of the Periodic Table,
p is a natural number from 0 to 10,
S is a fluorescent, universal base surrogate, able to intercalate into DNA,
M is a substituted carbon moiety of the CH or CR type, wherein R is hydrogen or an organic group, or
M is a substituted or unsubstituted amino group of the NH or NR type, wherein R is hydrogen or an organic group, or
M is an atom of the 5th or 6th main group of the Periodic Table,
Y is defined as A,
q is a natural number from 0-5,
r is a natural number from 0-5,
s is a natural number from 0 5.

2. A probe of the formula: 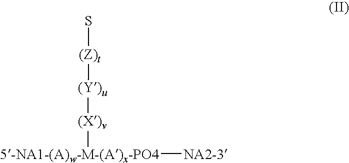
in which
5′-NA1 is a nucleic acid or a nucleic acid analog of any sequence and length, which ends in a 3′-terminal phosphate group,
NA2-3′ is a nucleic acid or a nucleic acid analog of any sequence and length,
A and A′ are independently of one another a methylene group or a substituted carbon moiety of the CHR or CR2 type, wherein R is hydrogen or an organic group,
w and x are a natural number from 0-5,
X′ and Z are independently of one another a methylene group or a substituted carbon moiety of the CHR or CR2 type, wherein R is hydrogen or an organic group, or
X′ and Z are independently of one another a substituted or unsubstituted amino group of the NH or NR type, wherein R is hydrogen or an organic group, or
X′ and Z are independently of one another an atom of the 5th or 6th main group of the Periodic Table,
Y′ is a methylene group or a substituted carbon moiety of the CHR or CR2 type, wherein R is hydrogen or an organic group, or
Y′ is a substituted or unsubstituted amino group of the NH or NR type, wherein R is hydrogen or an organic group rest, or
Y′ is an atom of the 5th or 6th main group of the Periodic Table,
v is a natural number from 0 to 10,
u is a natural number from 0 to 10,
t is a natural number from 0 to 10,
S is a fluorescent, universal base surrogate able to intercalate into DNA,
M is a substituted carbon moiety of the CH or CR type, wherein R is hydrogen or an organic group, or
M is a substituted or unsubstituted amino group of the NH or NR type, wherein R is hydrogen or an organic group, or
M is an atom of the 5th or 6th main group of the Periodic Table.

3. The probe as claimed in claim 1, which comprises a plurality of units of the formula (I) are covalently linked and has multiple labelings.

4. A process for preparing the probe as claimed in claim 1, comprising solid phase synthesis.

5-7. (canceled)

8. The probe as claimed in claim 1, wherein said probe comprises L-ornitine to which a fluorescent base surrogate is bound.

9. The probe as claimed in claim 1, wherein said probe comprises D-ornitine to which a fluorescent base surrogate is bound.

10. A test kit comprising the probe as claimed in claim 1 and at least one additional reagent.

11. The probe as claimed in claim 2, which comprises a plurality of units of the formula (II) covalently linked and has multiple labelings.

12. A process for preparing the probe as claimed in claim 2, comprising solid phase synthesis.

13. A method of assaying nucleic acid comprising incubating nucleic acid with a probe as claimed in claim 1.

14. A method of performing a PCR reaction comprising incubating nucleic acid with a probe as claimed in claim 1.

15. A method of performing a multiplex assay comprising incubating a plurality of probes according to claim 1 with nucleic acid, wherein different probes are labeled by different fluorescent base surrogates.

16. A method of assaying nucleic acid comprising incubating nucleic acid with a probe as claimed in claim 2.

17. A method of performing a PCR reaction comprising incubating nucleic acid with a probe as claimed in claim 2.

18. A method of performing a multiplex assay comprising incubating a plurality of probes according to claim 2 with nucleic acid, wherein different probes are labeled by different fluorescent base surrogates.

19. A test kit comprising the probe as claimed in claim 2 and at least one additional reagent.
